SEQUENCE LISTING (2 sequences)

SEQ ID NO: 1 - protein, 404 aa, synthetic construct
MATVNRSRHHHHHHHHIEGRSFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGKLAMTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 2 - DNA, 1215 bp, synthetic construct
  source 1..1215: mol_type = other DNA
atggcgacagtaaacagaagtcgtcatcaccaccatcatcatcaccacattgagggccgtagcttttcgcgccctggtcttcccgtagaatatttacaagtgcctagtccaagtatgggacgcgatataaaggtgcagttccagagcggtggtaataactcaccagctgtctacttacttgatgggttaagagcgcaggatgattacaatgggtgggacatcaacactcctgcatttgaatggtattaccaaagcggtctttcaatagtcatgcccgttggcggccaaagctcgttttactccgactggtacagccctgcgtgcggaaaggccggatgccagacttacaaatgggagacattcctgaccagcgaactgccacagtggttatccgcgaaccgcgccgtgaagccaactgggagtgcggctattggtttatctatggctggatcatcagcaatgatattggctgcctaccacccacagcaatttatttacgctggttccctgtcggccttgttggacccgagccaggggatggggccctctcttataggtttagcgatgggagacgcgggtggatataaagctgctgatatgtggggtccgtcctccgacccagcatgggagcggaacgatcccacccaacagattccgaagttagttgctaataacacacgcctgtgggtctattgtgggaatggtactcccaacgagctgggaggcgccaacattccagcagaattcttagagaacttcgttcgtagttctaacttgaaattccaagatgcttacaatgctgctggaggccataacgccgtttttaacttcccgccgaacggcacccactcatgggagtactggggcgcgcaattgaacgcgatgaagggtgacctgcaatcgagccttggtgccggcaaattagctatgacggaacaacaatggaattttgctggtatagaggctgcggcttcggcaatacaaggaaatgtaacctctatccatagccttttagatgaaggcaaacagtctttgacaaaacttgcggcagcttggggcggttctggtagtgaggcctatcagggcgtacagcagaagtgggacgctacggcgactgagcttaataatgcgttacagaatctggcccgcactatctccgaggctggtcaggcgatggcaagcaccgaaggcaacgttaccggaatgttcgcgtaa

(19) United States
(12) Patent Application Publication
(10) Pub. No.: US 2026/0306604 A1
(43) Pub. Date: Oct. 8, 2026

(54) MUCOSAL VACCINES AGAINST TUBERCULOSIS

(71) Applicant: UNIVERSITY OF HOUSTON SYSTEM, Houston, TX (US)

(72) Inventors: Navin Varadarajan, Houston, TX (US); K M Samiur Rahman Sefat, Houston, TX (US); Monish Kumar, Houston, TX (US)

(73) Assignee: UNIVERSITY OF HOUSTON SYSTEM, Houston, TX (US)

(21) Appl. No.: 19/474,900

(22) PCT Filed: Apr. 12, 2024
(86) PCT No.: PCT/US2024/024308

§ 371 Date: Oct. 13, 2025

Related U.S. Application Data
(60) Provisional application No. 63/458,698, filed on Apr. 12, 2023.

(51) Int. Cl. A61K 39/04; A61K 9/48; A61K 38/16; A61K 39/00; A61K 39/39; A61P 31/06

(52) U.S. Cl. CPC A61K 39/04; A61K 9/48; A61K 38/164; A61K 39/39; A61P 31/06; A61K 2039/53; A61K 2039/543; A61K 2039/55555; A61K 2039/572

(57) ABSTRACT

Embodiments of the present disclosure pertain to a composition that includes a particle, an antigen, and a modulator. In some embodiments, the modulator includes, without limitation, an agonist, an activator of the immune system, or combinations thereof. In some embodiments, the antigen is operable to elicit an immune response in a subject against Mycobacterium tuberculosis. Additional embodiments of the present disclosure pertain to methods of treating or preventing tuberculosis in a subject by administering a composition of the present disclosure to the subject.

### CROSS-REFERENCE TO RELATED APPLICATIONS

This application claims priority to U.S. Provisional Patent Application No. 63/458,698, filed on Apr. 12, 2023. The entirety of the aforementioned patent application is incorporated herein by reference.

### STATEMENT REGARDING FEDERALLY SPONSORED RESEARCH

This invention was made with government support under R01GM143243 awarded by the National Institutes of Health. The government has certain rights in the invention.

### SEQUENCE DISCLOSURE STATEMENT

Pursuant to 37 C.F.R. § 1.834, Applicant has submitted a sequence listing in XML format (“Sequence Listing”). The name of the file containing the Sequence Listing is “AF23853.P197W0.xml”. The date of the creation of the Sequence Listing is Apr. 12, 2024. The size of the Sequence Listing is 5,000 bytes. Applicant hereby incorporates by reference the material in the Sequence Listing.

### BACKGROUND

A need exists for the development and use of more effective vaccines against tuberculosis (TB). Numerous embodiments of the present disclosure aim to address the aforementioned need.

### SUMMARY

In some embodiments, the present disclosure pertains to a composition that includes a particle, an antigen, and a modulator. In some embodiments, the modulator includes, without limitation, an agonist, an activator of the immune system, or combinations thereof. In some embodiments, the antigen is operable to elicit an immune response in a subject against Mycobacterium tuberculosis.

In some embodiments, the modulator is encapsulated within the particle. In some embodiments, the modulator includes an endogenous agonist of the stimulator of interferon genes (STING) pathway. In some embodiments, the modulator includes 2′,3′-cyclic guanosine monophosphate-adenosine monophosphate (2′,3′-cGAMP).

In some embodiments, the antigen is associated with an outer surface of the particle. In some embodiments, the antigen includes a protein or peptide derived from Mycobacterium tuberculosis, or a nucleotide sequence encoding the protein or peptide.

In some embodiments, the antigen includes a fusion protein of Ag85b and ESAT6. In some embodiments, the fusion protein includes SEQ ID NO: 1 or a sequence with at least 65% sequence identity to SEQ ID NO: 1.

In some embodiments, the antigen includes a nucleotide sequence encoding a fusion protein of Ag85b and ESAT6. In some embodiments, the nucleotide sequence includes SEQ ID NO: 2 or a sequence with at least 65% sequence identity to SEQ ID NO: 2.

In some embodiments, the particle includes a lipid-based particle. In some embodiments, the lipid-based particle is in the form of a liposome. In some embodiments, the composition is suitable for use in treating or preventing tuberculosis.

Additional embodiments of the present disclosure pertain to methods of treating or preventing tuberculosis in a subject by administering a composition of the present disclosure to the subject. In some embodiments, the methods of the present disclosure are used to prevent tuberculosis in the subject. In some embodiments, the methods of the present disclosure are used to treat tuberculosis in the subject.

### BRIEF DESCRIPTION OF THE DRAWINGS

FIG. 1 provides a depiction of a composition that includes a particle, a modulator, and an antigen for treating or preventing tuberculosis, in accordance with various embodiments of the present disclosure.

FIGS. 2A and 2B illustrate methods of treating or preventing tuberculosis in a subject.

FIGS. 3A-3D illustrate the design and purification of H1 antigen (Ag85b-ESAT-6) in E. coli.

FIG. 3A shows the construct design for H1 antigen with a cleavable Sumo-tag for enhanced protein expression and solubility. FIG. 3B shows an SDS-page of the purified Sumo-H1 protein (56 kDa) and purified soluble H1 antigen (40 kDa). FIGS. 3C-3D show a physical characterization of NanoSTING (FIG. 3C) and NanoSTING-H1 (FIG. 3D) by dynamic light scattering (DLS).

FIGS. 4A-4D demonstrate that NanoSTING increases the persistence time of H1 antigens in the nasal cavity of vaccinated animals. FIG. 4A shows mouse schematics illustrating the nasal cavity (top) and the dorsal view of the mouse upper palate (bottom), including the region of interest (ROI) used to quantify fluorescent signals from IVIS images in FIGS. 4C and 4D. FIG. 4B shows an experimental scheme where mice were inoculated intranasally (IN) with NanoSTING-H1 iFluor 647 (20 μg NanoSTING and 10 μg H1 antigen), H1 Ag iFluor 647 (10 μg) or PBS. Biodistribution was visualized as a factor of light emission from iFluor 647 conjugated protein at different time points. FIG. 4C shows a representative IVIS images of fluorescence emitted by H1 antigen iFluor 647 in the nasal cavity of BALB/c mice (n=¾ animals per group) administered with different vaccine formulations. FIG. 4D shows quantified IVIS signals in the nasal cavity over 24 hours reported as radiant efficiency (p=photon). The signal calculated from nasal cavity ROI was background corrected by subtracting the signal from nasal-cavity-distal ROI for each mouse. Mann-Whitney t-test p values; ns=p>0.05, *p<0.05.

FIGS. 5A-5F show intranasal administration of NanoSTING-H1 induces robust T-cell response against multiple antigens in the lung and spleen of vaccinated animals. FIG. 5A shows the formulation of an intranasal vaccine, which was formulated by incubating purified recombinant protein and adjuvant. Applicant immunized three groups (n=5-10/group) of animals with intranasally NanoSTING-H1, H1 Ag, and PBS vaccine formulations. Four weeks after the first dose, the animals received an intranasal booster. FIGS. 5B and 5C show IFNγ ELISPOT from lung and spleen cells re-stimulated ex vivo with Ag85B and ESAT-6 peptide pools seven days after the second boost. Data are expressed as the mean (±SEM) of 10 animals assayed in two pools of five. Dunn's multiple comparison test p values: **p<0.01,***p<0.001,****p<0.0001. FIG. 5D shows ICS for the percentage of CD4⁺IL17 secreting T cells in the lung of mice 2 weeks after the booster. For p values, Applicant performed the Mann-Whitney t-test (*p<0.05). FIG. 5E shows ICS for the percentage of CD4⁺CXCR3⁺KLRG1⁻ T cells in the lung of mice 2 weeks after the booster. Mann-Whitney t-test p values; **p<0.01. FIG. 5F shows ICS for the percentage of CD4⁺CXCR3⁻KLRG1⁺ T cells in the lungs of mice 2 weeks after the booster. Mann-Whitney t-test p values; ns=p>0.05. The data in FIGS. 5D-5F are expressed as mean (±SEM), and each symbol represents an individual animal.

FIGS. 6A-6E show that intranasal vaccination with NanoSTING-H1 provides comparable protection to BCG against aerosolized Mtb strain Erdman. FIG. 6A illustrates the immunization of 26 C57BL/6 mice intranasally that followed the prime-boost regimen used in an immunogenicity study. A group (n=5) of mice were also immunized subcutaneously with BCG. Both groups of animals were then challenged with aerosolized Mtb strain Erdman. The animals were sacrificed four weeks after the challenge, and lungs and spleens were collected for CFU and flow cytometry assays. FIG. 6B shows colony forming unit (CFU) counts from mice three weeks after the challenge. Mann-Whitney t-test p values; ****p<0.0001. FIG. 6C shows percent bodyweight of mice compared to baseline at the indicated time intervals. Tukey's multiple comparison test p values; *p<0.05, **p<0.01,***p<0.001, ****p<0.0001. FIGS. 6D and 6E show ICS for percentage of Ag85B and ESAT-6 specific CD4+(FIG. 6D) and CD8+(FIG. 6E) T cells. Data is presented as mean (±SEM) of five animals assayed in each group. Mann-Whitney t-test p values; **p<0.01.

FIGS. 7A-7C show histopathological analysis of mouse lungs following Mtb challenge. FIGS. 7A-7B show mean lung histopathology scores three weeks (FIG. 7A) and 10 weeks (FIG. 7B) post-infection. Mann-Whitney test p values; ns=p>0.05, *p<0.05. FIG. 7C shows H&E staining of lung tissue from unvaccinated and non-infected (NI), unvaccinated and infected (PBS), BCG-vaccinated, and HI-vaccinated infected C57BL/6 mice at three- and 10-weeks post-challenge. Pictures are 20× enlargement showing the organized lymphocyte architecture among vaccinated (BCG and H1) mice absent in the PBS group and necrosis present in the PBS group later in disease that is prevented by vaccination.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components that include one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Tuberculosis (TB), caused by Mycobacterium tuberculosis (Mtb), is one of the oldest continuously recorded diseases in humans. Up to one in four people worldwide have been infected with TB and at least one million humans die every year from the disease. Despite this burden, an effective vaccine against TB does not exist.

Bacille Calmette-Guérin (BCG), the only vaccine against tuberculosis (TB), has a range of protective efficacy against adult pulmonary TB, ranging from 0% to 80% in clinical trials. However, since BCG is a live attenuated vaccine, BCG is not recommended for those with weakened immune systems, such as newborns and individuals infected with the human immunodeficiency virus (HIV).

As such, a need exists for the development and use of more effective vaccines against TB. Numerous embodiments of the present disclosure aim to address the aforementioned need.

### Compositions

In some embodiments, the present disclosure pertains to a composition. In some embodiments, the composition includes a particle. In some embodiments, the composition also includes a modulator. In some embodiments, the composition also includes an antigen. As set forth in more detail herein, the compositions of the present disclosure can include various types of particles, modulators, and antigens in various arrangements.

### Modulators

The compositions of the present disclosure can include various modulators. For instance, in some embodiments, the modulator includes, without limitation, an agonist, an activator of the immune system, or combinations thereof.

In some embodiments, the modulator is an agonist. In some embodiments, the agonist is a modulator of the stimulator of interferon genes (STING) pathway. In some embodiments, the agonist is an agonist of the stimulator of interferon genes (STING) pathway. In some embodiments, the agonist is an endogenous agonist of the stimulator of interferon genes (STING) pathway. In some embodiments, the modulator includes, without limitation, an endogenous agonist of the stimulator of interferon genes (STING) pathway, bis-(3′,5′)-cyclic dimeric guanosine monophosphate (c-di-GMP), cyclic guanosine monophosphate-adenosine monophosphate (cGAMP), 2′,3′-cyclic guanosine monophosphate-adenosine monophosphate (2′,3′-cGAMP), amidobenzimidazole, derivatives of amidobenzimidazole, nucleotide modulators, plasmid DNA modulators, CF501 (CAS No. 2408723-12-4), MSA-2 (CAS No. 129425-81-6), SHR1032 (structure disclosed in Chunying Song, et al. Sci Rep. 2022 May 20; 12(1):8579), C-178 (CAS No. 329198-87-0), H-151 (CAS No. 941987-60-6), divalent cations, or combinations thereof. In some embodiments, the modulator includes 2′,3′-cyclic guanosine monophosphate-adenosine monophosphate (2′,3′-cGAMP).

The modulators of the present disclosure may be associated with particles in various manners. For instance, in some embodiments, the modulators of the present disclosure are encapsulated within the particle. In some embodiments, the modulators of the present disclosure are on a surface of a particle.

In some embodiments, encapsulating one or more modulators within a particle as described herein can increase uptake efficiency, tissue targeting, stability, and/or efficacy of the composition or a method of its use, such as by controlling (e.g., maintaining) spatial concentration of the one or more modulators and/or by presenting a recognition signal (e.g., an antigen) to a target cell. In some embodiments, compositions that include one or more modulators without a particle or antigen (or that include one or more modulators outside of and unassociated with a particle of the composition) can elicit a more immediate response from a target tissue or cell and/or decrease the cost and/or complexity of manufacture of the composition.

The compositions of the present disclosure can include various amounts of modulators. For instance, in some embodiments, a composition of the present disclosure can include 0.1 micrograms to 200 micrograms of a modulator (e.g., a STING pathway activator (e.g., a STING agonist)).

### Antigens

The compositions of the present disclosure can also include various antigens. For instance, in some embodiments, the antigen is operable to elicit an immune response in a subject against Mycobacterium tuberculosis. In some embodiments, the antigen includes a protein or peptide derived from Mycobacterium tuberculosis (e.g., recombinant proteins or peptides), or a nucleotide sequence encoding the protein or peptide (e.g., a plasmid DNA molecule and/or minigenes expressing the protein or peptide). In some embodiments, the antigen includes a protein or peptide derived from Mycobacterium tuberculosis. In some embodiments, the antigen includes a nucleotide sequence encoding a protein or peptide derived from Mycobacterium tuberculosis.

In some embodiments, the antigen includes a protein derived from Mycobacterium tuberculosis, or a nucleotide sequence encoding the protein. In some embodiments, the protein is in the form of a fusion protein, a protein fragment, a full protein, or combinations thereof.

In some embodiments, the antigen includes, without limitation, Ag85b, ESAT6, Rv1813, Rv1733c, RV2626c, Rv2660c, RpfD, Ag85A, CFP10, TB10.4, EsxV, EsxW, MTB32A, MTB39A, PPE42, a full protein thereof, a protein fragment thereof, a peptide fragment thereof, a fusion protein thereof, or combinations thereof. In some embodiments, the antigen includes one or more of Ag85b, ESAT6 and Rv1813 proteins. In some embodiments, the antigen includes one or more of Ag85b, ESAT6, Rv1733c, RV2626c and RpfD. In some embodiments, the antigen includes one or more of Ag85A and CFP10. In some embodiments, the antigen includes one or more of TB10.4, EsxV, and EsxW. In some embodiments, the antigen includes one or more of MTB32A, MTB39A and PPE42.

In some embodiments, the antigen includes a fusion protein of Mycobacterium tuberculosis, or a nucleotide encoding the fusion protein. In some embodiments, the fusion protein includes, without limitation, a fusion of two or more of Ag85b, ESAT6, Rv1813, Rv1733c, RV2626c, Rv2660c, RpfD, Ag85A, CFP10, TB10.4, EsxV, EsxW, MTB32A, MTB39A, PPE42, a full protein thereof, a protein fragment thereof, a peptide fragment thereof, or combinations thereof. In some embodiments, the fusion protein includes, without limitation, a fusion of Ag85b and ESAT6, an H56 fusion protein (Ag85B-ESAT6-Rv2660c) or combinations thereof.

In some embodiments, the antigen is a fusion protein of Ag85b and ESAT6. In some embodiments, the fusion protein includes SEQ ID NO: 1. In some embodiments, the fusion protein includes a sequence with at least 65% sequence identity to SEQ ID NO: 1. In some embodiments, the fusion protein includes a sequence with at least 70% sequence identity to SEQ ID NO: 1. In some embodiments, the fusion protein includes a sequence with at least 75% sequence identity to SEQ ID NO: 1. In some embodiments, the fusion protein includes a sequence with at least 80% sequence identity to SEQ ID NO: 1. In some embodiments, the fusion protein includes a sequence with at least 85% sequence identity to SEQ ID NO: 1. In some embodiments, the fusion protein includes a sequence with at least 90% sequence identity to SEQ ID NO: 1. In some embodiments, the fusion protein includes a sequence with at least 95% sequence identity to SEQ ID NO: 1. In some embodiments, the fusion protein includes a sequence with at least 99% sequence identity to SEQ ID NO: 1.

In some embodiments, the antigen is a nucleotide sequence encoding a fusion protein of Ag85b and ESAT6. In some embodiments, the nucleotide sequence includes SEQ ID NO: 2. In some embodiments, the nucleotide sequence includes a sequence with at least 65% sequence identity to SEQ ID NO: 2. In some embodiments, the nucleotide sequence includes a sequence with at least 70% sequence identity to SEQ ID NO: 2. In some embodiments, the nucleotide sequence includes a sequence with at least 75% sequence identity to SEQ ID NO: 2. In some embodiments, the nucleotide sequence includes a sequence with at least 80% sequence identity to SEQ ID NO: 2. In some embodiments, the nucleotide sequence includes a sequence with at least 85% sequence identity to SEQ ID NO: 2. In some embodiments, the nucleotide sequence includes a sequence with at least 90% sequence identity to SEQ ID NO: 2. In some embodiments, the nucleotide sequence includes a sequence with at least 95% sequence identity to SEQ ID NO: 2. In some embodiments, the nucleotide sequence includes a sequence with at least 99% sequence identity to SEQ ID NO: 2.

The antigens of the present disclosure may be associated with the particles of the present disclosure in various manners. For instance, in some embodiments, the antigen is associated with an outer surface of the particle. In some embodiments, the antigen is encapsulated within the particle.

In some embodiments, association of an antigen with a particle (e.g., on an outer surface of a particle) that is associated with a modulator (e.g., an encapsulated modulator) can significantly increase the targeting and/or delivery of the modulator to a target tissue of interest (e.g., an intranasal compartment and/or a lung compartment of a subject). In some embodiments, the association of an antigen with a particle of the present disclosure may increase the efficacy of the composition in treating and/or preventing tuberculosis in a subject, such as by spatially concentrating an antigen and a modulator of the composition (e.g., at a target tissue).

The compositions of the present disclosure can include various amounts of antigens. For instance, in some embodiments, a composition of the present disclosure can include 0.1 micrograms to 200 micrograms of an antigen.

### Particles

The compositions of the present disclosure can include various particles. For instance, in some embodiments, the particles include, without limitation, a lipid-based particle, a carbon-based particle, a metal-based particle, or combinations thereof. In some embodiments, the particles of the present disclosure include a lipid-based particle. In some embodiments, the lipid-based particle is in the form of a liposome. In some embodiments, the lipid-based particle includes one or more lipids. In some embodiments, the one or more lipids include, without limitation, 1,2-dipalmitoyl-sn-glycero-3-phosphocholine (DPPC), 1,2-dipalmitoyl-sn-glycero-3-phospho-(1′-rac-glycerol) (DPPG), cholesterol, 1,2-dipalmitoyl-sn-glycero-3-phosphoethanolamine-N-[methoxy(polyethylene glycol)-2000] DPPE-PEG2000, or combinations thereof. In some embodiments, the lipid-based particle includes DPPC, DPPG, DPPE-PEG2000 at a molar ratio of 10:1:1:1.

In some embodiments, a particle can include a membrane or wall. In some embodiments, a membrane or wall of a particle can define an interior space. In some embodiments, an interior space of a particle can include one or more modulators. In some embodiments, one or more antigens can be associated with a membrane or wall of the particle (e.g., an outer surface of a membrane or wall of the particle).

In some embodiments, the particles of the present disclosure include a plurality of molecules. In some embodiments, the particles of the present disclosure or a portion thereof can be anionic (e.g., can include an anionic membrane or one or more anionic lipids). For example, in some embodiments, a lipid-based particle of the present disclosure can include an anionic lipid (e.g., DPPE, DPPE-PEG2000, DPPC, DPPG) or a neutral lipid (e.g., cholesterol). In some embodiments, a particle of the present disclosure may include no cationic lipids. In some embodiments, a particle of the present disclosure can be cationic.

In some embodiments, a particle of the present disclosure can include a cationic (e.g., positively charged) lipid. For example, in some embodiments, a particle of the present disclosure can include, without limitation, 1,2-dipalmitoyl-3-trimethylammonium-propane chloride (DPTAP) or 1,2-dioleoyl-3-trimethylammonium propane (DOTAP).

In some embodiments, a particle of the present disclosure can be zwitterionic. In some embodiments, a particle of the present disclosure or a portion thereof can have a net zero charge. In some embodiments, a particle of the present disclosure or a portion thereof can be uncharged. In some embodiments, a particle of the present disclosure can include dipalmitoylphosphatidylcholine, dipalymitoylphosphatidylglycerol, 1,2-bis(diphenylphosphino)ethane (DPPE), cholesterol, or a combination thereof.

In some embodiments, a particle of the present disclosure can include a poly(ethyleneglycol)-lipid (e.g., a PEG-lipid). In some embodiments, a particle of the present disclosure can include DPPC (1,2-dipalmitoyl-sn-glycero-3-phosphocholine), DPPG (1,2-dipalmitoyl-sn-glycero-3-phospho-(1′-rac-glycerol)), DPPE-PEG2000 (1,2-dipalmitoyl-sn-glycero-3-phosphoethanolamine-N-[methoxy(polyethylene glycol)-2000]), cholesterol, 1,2-dipalmitoyl-3-trimethylammonium-propane chloride (DPTAP), 1,2-dioleoyl-3-trimethylammonium propane (DOTAP), or a combination thereof.

In some embodiments, a particle of the present disclosure can include a combination of DPPC and DPPG, for example, at a molar ratio of about 10:1. In some embodiments, a particle of the present disclosure can include a combination of DPPC and cholesterol, for example, at a molar ratio of about 10:1. In some embodiments, a particle of the present disclosure can include a combination of DPPC and DPPE-2000, for example, at a molar ratio of about 10:1. In some embodiments, a particle of the present disclosure can include a combination of DPPG and cholesterol, for example, at a molar ratio of about 1:1. In some embodiments, a particle of the present disclosure can include a combination of cholesterol and DPPE-PEG2000, for example, at a molar ratio of about 1:1. In some embodiments, a particle of the present disclosure can include a combination of DPPG and DPPE-PEG2000, for example, at a molar ratio of about 1:1.

In some embodiments, a particle of the present disclosure can include DPPC, DPPG, cholesterol, and DPPE-PEG2000. In some embodiments, a particle of the present disclosure can be composed of a molar ratio of 10:1:1:1 of DPPC, DPPG, cholesterol, and DPPE-PEG2000, respectively. In some embodiments, a particle of the present disclosure can be composed of a molar ratio of 20:1:1:1 of DPPC, DPPG, cholesterol, and DPPE-PEG2000, respectively. In some embodiments, a particle of the present disclosure can be composed of a molar ratio of 5:1:1:1 of DPPC, DPPG, cholesterol, and DPPE-PEG2000, respectively. In some embodiments, a particle of the present disclosure can be composed of a molar ratio of 10:2:1:1 of DPPC, DPPG, cholesterol, and DPPE-PEG2000, respectively. In some embodiments, a particle of the present disclosure can be composed of a molar ratio of 10:1:2:1 of DPPC, DPPG, cholesterol, and DPPE-PEG2000, respectively.

In some embodiments, a particle of the present disclosure can be composed of a molar ratio of 10:1:1:2 of DPPC, DPPG, cholesterol, and DPPE-PEG2000, respectively. In some embodiments, a particle of the present disclosure can be composed of a molar ratio of 10:2:2:1 of DPPC, DPPG, cholesterol, and DPPE-PEG2000, respectively. In some embodiments, a particle of the present disclosure can be composed of a molar ratio of 10:1:2:2 of DPPC, DPPG, cholesterol, and DPPE-PEG2000, respectively. In some embodiments, a particle of the present disclosure can be composed of a molar ratio of 10:2:1:2 of DPPC, DPPG, cholesterol, and DPPE-PEG2000, respectively.

In some embodiments, the particles of the present disclosure can include a nanoparticle. In some embodiments, the nanoparticles have particle diameter (e.g., mean hydrodynamic particle diameter) of less than 300 nanometers (nm), less than 200 nm, less than 150 nm, less than 120 nm, less than 115 nm, less than 111 nm, less than 110 nm, less than 105 nm, less than 100 nm, less than 95 nm, less than 90 nm, less than 85 nm, or less than 80 nm.

In some embodiments, the particles of the present disclosure have an outer diameter of 1 nanometer to 500 nanometers, 1 nanometer to 750 nanometers, or 1 nanometer to 1,000 nanometers. In some embodiments, the particles of the present disclosure can have an outer diameter of 1 nanometer to 10 nanometers, 1 nanometer to 15 nanometers, 1 nanometer to 20 nanometers, 1 nanometer to 30 nanometers, 1 nanometer to 50 nanometers, 1 nanometer to 75 nanometers, 1 nanometer to 100 nanometers, 1 nanometer to 150 nanometers, 1 nanometer to 200 nanometers, 1 nanometer to 250 nanometers, 1 nanometer to 300 nanometers, 1 nanometer to 400 nanometers, 1 nanometer to 500 nanometers, 10 nanometers to 15 nanometers, 10 nanometers to 20 nanometers, 10 nanometers to 30 nanometers, 10 nanometers to 50 nanometers, 10 nanometers to 75 nanometers, 10 nanometers to 100 nanometers, 10 nanometers to 150 nanometers, 10 nanometers to 200 nanometers, 10 nanometers to 250 nanometers, 10 nanometers to 300 nanometers, 15 nanometers to 20 nanometers, 15 nanometers to 30 nanometers, 15 nanometers to 50 nanometers, 15 nanometers to 75 nanometers, 15 nanometers to 100 nanometers, 15 nanometers to 150 nanometers, 15 nanometers to 200 nanometers, 15 nanometers to 250 nanometers, 15 nanometers to 300 nanometers, 20 nanometers to 30 nanometers, 20 nanometers to 50 nanometers, 20 nanometers to 75 nanometers, 20 nanometers to 100 nanometers, 20 nanometers to 150 nanometers, 20 nanometers to 200 nanometers, 20 nanometers to 250 nanometers, 20 nanometers to 300 nanometers, 30 nanometers to 50 nanometers, 30 nanometers to 75 nanometers, 30 nanometers to 100 nanometers, 30 nanometers to 150 nanometers, 30 nanometers to 200 nanometers, 30 nanometers to 250 nanometers, 30 nanometers to 300 nanometers, 50 nanometers to 75 nanometers, 50 nanometers to 100 nanometers, 50 nanometers to 150 nanometers, 50 nanometers to 200 nanometers, 50 nanometers to 250 nanometers, 50 nanometers to 300 nanometers, 75 nanometers to 100 nanometers, 75 nanometers to 150 nanometers, 75 nanometers to 200 nanometers, 75 nanometers to 250 nanometers, 75 nanometers to 300 nanometers, 100 nanometers to 150 nanometers, 100 nanometers to 200 nanometers, 100 nanometers to 250 nanometers, 100 nanometers to 300 nanometers, 150 nanometers to 200 nanometers, 150 nanometers to 250 nanometers, 150 nanometers to 300 nanometers, 200 nanometers to 250 nanometers, 200 nanometers to 300 nanometers, or 250 nanometers to 300 nanometers. In some embodiments, a particle can have an outer diameter of 1 nanometer, 10 nanometers, 15 nanometers, 20 nanometers, 30 nanometers, 50 nanometers, 75 nanometers, 100 nanometers, 150 nanometers, 200 nanometers, 250 nanometers, 300 nanometers, 400 nanometers, or 500 nanometers.

In some embodiments, the particles of the present disclosure have an outer diameter of at least 1 nanometer, 10 nanometers, 15 nanometers, 20 nanometers, 30 nanometers, 50 nanometers, 75 nanometers, 100 nanometers, 150 nanometers, 200 nanometers, 250 nanometers, 300 nanometers, 400 nanometers, or 500 nanometers. In some embodiments, the particles of the present disclosure can have an outer diameter of at most 1 nanometer, 10 nanometers, 15 nanometers, 20 nanometers, 30 nanometers, 50 nanometers, 75 nanometers, 100 nanometers, 150 nanometers, 200 nanometers, 250 nanometers, 300 nanometers, 400 nanometers, 500 nanometers, 750 nanometers, or 1,000 nanometers.

A modulator and/or an antigen of the present disclosure may be associated with the particles of the present disclosure in various manners. For instance, in some embodiments, the antigens and modulators of the present disclosure can be positioned on different regions of the particles of the present disclosure. In some embodiments, a modulator of the present disclosure can be encapsulated in a particle. In some embodiments, all or a portion of an antigen of the present disclosure can be associated with (e.g., attached to, adhered to, adsorbed onto, electrostatically interacted with, covalently bound to, noncovalently bound to, integrated into, or formulated onto) a surface of the particle. In some embodiments, all or a portion of an antigen is encapsulated within the lipid-based particle. In some embodiments, the association (e.g., adsorption) of an antigen with a surface of a particle can increase stability of the particle and/or increase delivery efficiency (e.g., to a target tissue) after administration.

In some embodiments, one or more antigens of the present disclosure can be encapsulated within a particle of the present disclosure. In some embodiments, one or more modulators of the present disclosure can be associated with an outer surface of the particle. In some embodiments, one or more antigens of the present disclosure can be encapsulated in a particle of the present disclosure while one or more modulators of the present disclosure are associated with an outer surface of the particle. In some embodiments, one or more antigens of the present disclosure can be associated with an outer surface of a particle of the present disclosure. In some embodiments, one or more modulators of the present disclosure can be encapsulated within the particles of the present disclosure.

In some embodiments, one or more antigens of the present disclosure can be associated with an outer surface of a particle of the present disclosure while one or more modulators of the present disclosure are encapsulated within the particle. In some embodiments, one or more antigens and one or more modulators of the present disclosure can both be encapsulated within a particle of the present disclosure. In some embodiments, one or more antigens and one or more modulators of the present disclosure may both be associated with a surface of a particle of the present disclosure. In some embodiments, one or more antigens of the present disclosure can be integrated into a membrane of a particle of the present disclosure. In some embodiments, one or more modulators of the present disclosure can be integrated into a membrane of a particle of the present disclosure. In some embodiments, one or more antigens of the present disclosure can be encapsulated within a particle of the present disclosure while one or more modulators are integrated into a membrane of a particle of the present disclosure. In some embodiments, one or more antigens of the present disclosure can be associated with an outer surface of a particle of the present disclosure while one or more modulators are integrated into a membrane of a particle of the present disclosure.

In some embodiments, one or more modulators of the present disclosure can be encapsulated within a particle of the present disclosure while one or more antigens of the present disclosure are integrated into a membrane of the particle. In some embodiments, one or more modulators can be associated with an outer surface of a particle of the present disclosure while one or more antigens of the present disclosure are integrated into a membrane of the particle. In some embodiments, the incorporation of both an antigen and a modulator of the present disclosure within a particle can facilitate coordinated cytosolic delivery.

The compositions of the present disclosure can include various amounts of particles. For instance, in some embodiments, a composition of the present disclosure can include 0.1 micrograms to 200 micrograms of a particle of the present disclosure.

### Composition Forms

The compositions of the present disclosure may be in various forms. For instance, in some embodiments, the compositions of the present disclosure may be formulated for intranasal delivery. In some embodiments, the composition is lyophilized. In some embodiments, the composition is in liquid form. In some embodiments, the composition is suitable for use in treating or preventing tuberculosis.

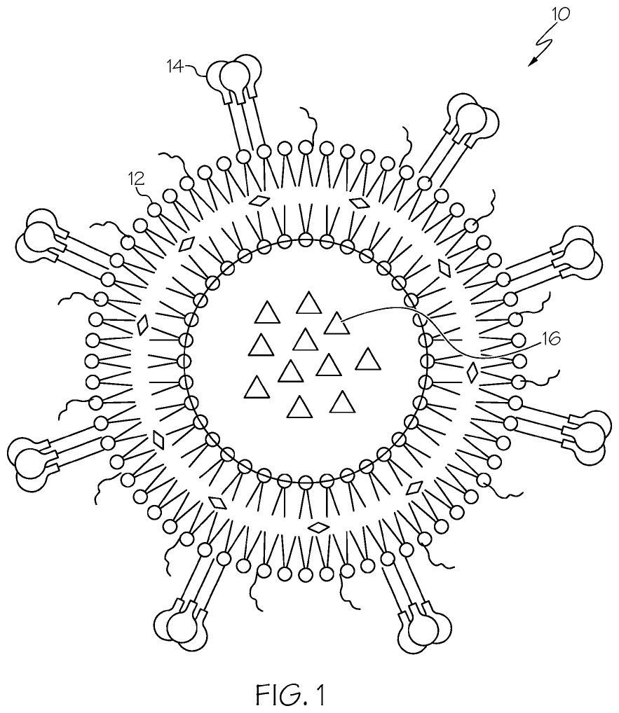

An example of a composition of the present disclosure is illustrated in FIG. 1 as composition 10, which includes a lipid-based particle 12 (e.g., a liposome), a modulator 16 (e.g., a STING agonist, such as cGAMP, and an antigen 14 (e.g., a fusion protein of Mycobacterium tuberculosis, such as a fusion protein of Ag85b and ESAT6). In this example, modulator 16 is encapsulated within lipid-based particle 12 while antigen 14 is associated with the outer surface of lipid-based particle 12.

In some embodiments, it can be advantageous to formulate the compositions of the present disclosure within a desired pH range. For example, formulation of a composition described herein (e.g., for intranasal delivery) between pH 4.0 and pH 7.5 (e.g., pH 4.5 to pH 6.5 or pH 5.5 to pH 6.5) can avoid irritation and/or histological damage to the intranasal tissue, which may occur if extremely acidic or basic formulations are used for intranasal delivery (e.g., because nasal cavity pH can be from about 5.5 to 6.5). In some embodiments, a composition of the present disclosure can have a pH of 4.0 to 7.5.

In some embodiments, it can be advantageous to formulate a composition of the present disclosure within a desired osmolarity range. For example, formulation of a composition of the present disclosure (e.g., for intranasal delivery) having an osmolarity from 50 to 900 mOsm/kg can improve absorption while avoiding potential epithelial damage (e.g., which may occur from compositions with very low osmolarities) and avoiding increases to mucosal secretions (e.g., which may occur from compositions with very high osmolarities) (e.g., because nasal cavity osmolarity can be about 280 mOsm/kg). In some embodiments, a composition of the present disclosure can have an osmolarity of 50 mOsm/kg to 900 mOsm/kg.

In some embodiments, the compositions of the present disclosure can be advantageous to formulate compositions described herein within a desired viscosity range. For example, formulation of a composition of the present disclosure (e.g., for intranasal delivery) having a viscosity, for example, from 1.1 cP (centipoise) to 50 cP (e.g., 1.5 cP to 50 cP) can increase residence time in the nasal cavity while adversely affecting droplet size (e.g., which can affect spray pattern and/or distribution within the nasal cavity). In some embodiments, a composition of the present disclosure can have a viscosity of 1 cP (centipoise) to 100 cP.

The compositions of the present disclosure can be in various forms. For instance, in some embodiments, the compositions of the present disclosure can be in the form of a solubilized liquid. In some embodiments, the compositions of the present disclosure are suitable for intranasal and/or inhalational administration to a subject. In some embodiments, intranasal delivery of the compositions of the present disclosure can be used to target intranasal compartment tissues. In some embodiments, inhalational administration of the compositions of the present disclosure can be used to target lung compartment tissues.

In some embodiments, the compositions of the present disclosure also include one or more stabilizers. In some embodiments, the stabilizers include, without limitation, anti-oxidants, sequestrants, ultraviolet stabilizers, or combinations thereof.

In some embodiments, the compositions of the present disclosure also include one or more surfactants. In some embodiments, the surfactants include, without limitation, anionic surfactants, sugars, cationic surfactants, zwitterionic surfactants, non-ionic surfactants, or combinations thereof.

In some embodiments, the compositions of the present disclosure also include one or more excipients. In some embodiments, the excipients include, without limitation, lactose, sucrose, starch powder, cellulose esters of alkanoic acids, trehalose, cellulose alkyl esters, talc, stearic acid, magnesium stearate, magnesium oxide, sodium and calcium salts of phosphoric and sulfuric acids, gelatin, acacia gum, trehalose, sodium alginate, polyvinylpyrrolidone, polyvinyl alcohol, or combinations thereof.

In some embodiments, a composition of the present disclosure is in lyophilized form (e.g., freeze-dried). Lyophilization of a composition of the present disclosure (or a component thereof) can increase the storage stability (e.g., shelf stability) of a composition. In some embodiments, one or more components of a composition of the present disclosure can be lyophilized.

In some embodiments, lyophilization of a composition of the present disclosure (or a component thereof) can allow for easy preparation of the composition. In some embodiments, such lyophilization allows for use of the compositions of the present disclosure (e.g., administration to a subject) in regions without easy access to material preparation facilities. For instance, a modulator, a lipid-based particle, and/or an antigen described herein can be lyophilized and then rehydrated and mixed (e.g., as described herein) to formulate a composition described herein at a site of administration to a subject that is remote from a permanent medical or pharmaceutical facility.

In some embodiments, a lyophilized composition of the present disclosure (or a portion thereof, such as a lyophilized particle or a lyophilized antigen or a lyophilized modulator) can be stored at 4° C. In some embodiments, a lyophilized composition of the present disclosure (or a portion thereof) can be stored at 4° C. and used for up to 1 week, up to 2 weeks, up to 3 weeks, up to 1 month, up to 2 months, up to 3 months, up to 4 months, up to 5 months, up to 6 months, up to 7 months, up to 8 months, up to 9 months, up to 10 months, or up to 12 months.

In some embodiments, the compositions of the present disclosure may be in liquid form. In some embodiments, the compositions of the present disclosure may be in solid form.

In some embodiments, a composition of the present disclosure can include a divalent ion, such as a divalent cation. In some embodiments, a composition of the present disclosure includes a divalent cation encapsulated within, adsorbed onto, covalently coupled to, electrostatically interacted with, or formulated onto a membrane of a particle of the present disclosure. In some embodiments, a composition can include a divalent cation that includes, without limitation, Mn²⁺, Mg²⁺, Ca²⁺, and Zn²⁺. For example, a composition of the present disclosure can include a modulator (e.g., a STING agonist), a particle (e.g., a lipid-based nanoparticle), and a divalent cation, such as Mn²⁺, Mg²⁺, Ca²⁺, or Zn²⁺.

### Methods of Treating or Preventing Tuberculosis

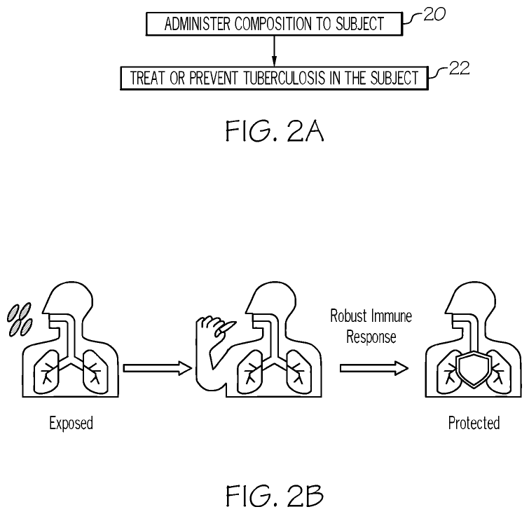

Additional embodiments of the present disclosure pertain to methods of treating or preventing tuberculosis in a subject. As illustrated in FIG. 2A, such methods generally include administering a composition of the present disclosure to the subject (step 20) to result in the treatment or prevention of tuberculosis in the subject (step 22). As set forth in more detail herein, the methods of the present disclosure can have numerous uses and embodiments.

### Subjects

The methods of the present disclosure may be utilized to treat or prevent tuberculosis in various subjects. For instance, in some embodiments, the subject does not exhibit symptoms of tuberculosis. In some embodiments, the subject exhibits symptoms of tuberculosis.

The compositions of the present disclosure may be administered to various subjects. For instance, in some embodiments, the subject is a mammal (e.g., a human). In some embodiments, the subject is a human being. In some embodiments, the subject is a domesticated animal. For example, in some embodiments, the subject can be a dog or a cat. In some embodiments, a subject can be a cow, a horse, a non-human primate, a mouse, a rat, a rabbit, a guinea pig, a goat, a sheep, a giraffe, a zebra, a lion, a tiger, or a bear.

In some embodiments, the subject can be vulnerable to or suffering from tuberculosis. For example, in some embodiments, the compositions of the present disclosure may be administered to a subject who has been exposed to or who has been infected with Mycobacterium tuberculosis. In some embodiments, a subject can be selected for treatment as a result of exhibiting one or more symptoms of tuberculosis. For example, a subject may be selected by for treatment based on having one or more symptoms, including persistent coughing, elevated body temperature (e.g., greater than 100.4° C. by forehead skin measurement), body chills, achy joints, difficulty breathing or catching one's breath, fluid in the lungs, fatigue, headache, loss of taste or smell, or a positive tuberculosis test, such as a PCR test.

In some embodiments, a subject may be a subject vulnerable to tuberculosis. For instance, in some embodiments, the subject may be selected for treatment with a composition of the present disclosure based on a demographic risk factor, such as obesity, advanced age (e.g., 65 years old or older), immune impairment, or pregnancy. In some embodiments, a subject may be selected for treatment based on a risk of infection by Mycobacterium tuberculosis, for instance, if the subject has an occupation involving close interaction with customers, frequent interaction with at-risk populations, handling of biological samples, or close contact with potentially infected individuals.

### Administration

The compositions of the present disclosure may be administered to subjects in various manners. For instance, in some embodiments, the composition is administered in at least one dose.

In some embodiments, the composition is administered in one dose. In some embodiments, the composition is administered in two doses.

The compositions of the present disclosure may be administered to subjects by various methods. For instance, in some embodiments, the administration occurs by methods that include, without limitation, intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, or combinations thereof. In some embodiments, the compositions of the present disclosure are administered through intranasal administration. In some embodiments, the compositions of the present disclosure are administered through inhalational administration.

In some embodiments, a composition of the present disclosure may be administered to a subject before exposure to Mycobacterium tuberculosis, for example, to prevent the subject from acquiring tuberculosis (e.g., as illustrated in FIG. 2A). In some embodiments, a composition of the present disclosure may be administered in the form of a vaccine.

In some embodiments, a method of the present disclosure can include administering a dose of a composition of the present disclosure (e.g., via intranasal administration) to a subject (e.g., an animal subject) after exposure or suspected exposure to Mycobacterium tuberculosis. In some embodiments, a composition of the present disclosure may be administered to a subject after exposure to Mycobacterium tuberculosis, for example, to treat (e.g., ameliorate or, in some embodiments, cure) tuberculosis, for example, after the subject has acquired tuberculosis (e.g., as illustrated in FIG. 2B). In some embodiments, the compositions of the present disclosure may be administered to a subject at least 1 minute, at least 5 minutes, at least 10 minutes, at least 15 minutes, at least 20 minutes, at least 30 minutes, at least 45 minutes, at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after exposure or suspected exposure to Mycobacterium tuberculosis.

In some embodiments, the compositions of the present disclosure can be administered in combination with other therapeutic treatments. In some embodiments, the other therapeutic treatments include, without limitation, antibiotic treatment, targeted inhibition, or combinations thereof. In some embodiments, a composition of the present disclosure can include and/or can be administered in a treatment regimen (e.g., administered concurrently or non-concurrently) with an adjuvant (e.g., one or more antibodies, one or more vaccines, one or more small molecules, one or more nucleic acids, and/or one or more peptides or proteins, such as an interleukin (e.g., IL-21)).

### Treatment or Prevention of Tuberculosis

The methods of the present disclosure may have various uses. For instance, in some embodiments, the methods of the present disclosure may be used to treat tuberculosis in a subject. In some embodiments, the methods of the present disclosure may be used to prevent tuberculosis in a subject. For instance, in some embodiments, the methods of the present disclosure may be used to prevent the establishment of tuberculosis in the subject, to prevent progression of tuberculosis in the subject, to prevent the transmission of tuberculosis to a second subject, or combinations thereof. In some embodiments, the methods of the present disclosure may initiate an innate immune response that leads to associated adaptive immunity for tuberculosis.

In some embodiments, the administered compositions of the present disclosure can rapidly activate a subject's innate immune system to facilitate a broad-spectrum response against Mycobacterium tuberculosis. For instance, in some embodiments, a single intranasal dose of a composition of the present disclosure can: (1) treat infections throughout the respiratory tree and minimize symptoms, (2) reduce transmission by decreasing infectious in the nasal passage, (3) protect against Mycobacterium tuberculosis, and/or (4) provide durable protection against reinfection by stimulating adaptive immunity.

In some embodiments, administered compositions of the present disclosure can enable sustained release of modulators (e.g., cGAMP) to both the nasal compartment and the lung over a period of 48 hours or more following administration to a subject. In some embodiments, the administered compositions of the present disclosure can activate multiple pathways and facilitate a type-I interferon (IFN-I) mediated response in a subject.

In some embodiments, the compositions of the present disclosure can elicit an immune response in the subject against tuberculosis. In some embodiments, the compositions of the present disclosure can elicit such immunity through at least one of innate immunity, mucosal immunity, systemic immunity, cellular immunity, humoral immunity, T-cell immunity, production of systemic neutralizing antibodies, induction of IgG responses, induction of IgA responses, induction of IgM responses, induction of T-cell responses, induction of mucosal IgA responses in lung and nasal compartments, induction of Th1 T-cell responses, induction of CD8+ T-cell responses, induction of CD4+ T cell responses, induction of NK cell responses, activation or inhibition of the stimulator of interferon genes (STING) pathway, or combinations thereof.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. An Intranasal Nanoparticle Vaccine Elicits Protective Immunity Against Mycobacterium tuberculosis

The development of vaccines that can either supplement or replace the live attenuated Mycobacterium bovis vaccine strain Bacille Calmette-Guérin (BCG) to provide a protective immune response against pulmonary tuberculosis (TB) has received much attention. One advantage of subunit vaccinations is that they typically have better safety profiles than live attenuated vaccines, which cannot always be administered to immunocompromised individuals.

An adjuvant is recommended to elicit a robust memory immune response to the antigen in subunit vaccines. However, there is a lack of authorized adjuvants that can safely induce antigen-specific effector and memory CD4⁺ and CD8⁺ T cells.

Moreover, a mucosal immune response is desired for preventing respiratory infections, such as TB. Dendritic cells (DCs), which transport the vaccinated antigen to local and systemic inductive sites such as the lymph nodes and spleen, cause both mucosal and systemic reactions due to mucosal vaccination. The widespread use of several effective mucosal vaccinations against non-respiratory pathogens such as polio, cholera, rotavirus, and salmonella has demonstrated the viability and safety of this strategy.

Cyclic dinucleotide, 2′-3′ cyclic GMP-AMP (cGAMP), has emerged recently as a potent adjuvant that can be co-delivered with subunit proteins and is effective in enhancing humoral, cellular, and mucosal immune responses. cGAMP binds to the stimulator of interferon genes (STING), leading to the oligomerization of STING and its translocation to the trans-Golgi network, initiating a signaling cascade that culminates in the secretion of type I and type III interferons. Due to its immunostimulatory role, cGAMP is being investigated as an immunotherapeutic for treating cancers and a mucosal adjuvant for developing vaccines against infectious diseases.

STING activating cyclic dinucleotide adjuvant used in a mucosal protein subunit vaccine against TB elicited both Th1 and Th17 immune responses in mice. An ESAT-6-based c-di-AMP adjuvanted mucosal vaccine decreased bacterial load in the lungs of challenged animals. When taken as a whole, these data offer a strong case for using STING agonists as a clinical TB vaccine adjuvant.

In this Example, Applicant tested NanoSTING, a liposomal formulation of the endogenous activator of the stimulator of interferon genes (STING) pathway, cyclic guanosine adenosine monophosphate (cGAMP), as a mucosal adjuvant to elicit protective immunity against Mycobacterium tuberculosis. Applicant formulated a vaccine based on the H1 antigen (fusion protein of Ag85b and ESAT6) adjuvanted with NanoSTING.

Intranasal immunization of NanoSTING-H1 elicited a strong T-cell response in the lung of vaccinated animals characterized by (a) CXCR3⁺KLRG1⁻ lung resident T cells that are known to be essential for controlling bacterial infection, (b) IFNγ-secreting CD4⁺ T cells which is necessary for intracellular bactericidal activity, and (c) IL17-secreting CD4⁺ T cells that can confer protective immunity against multiple clinically relevant strains of Mtb. Upon challenge with aerosolized Mycobacterium tuberculosis Erdman strain, intranasal NanoSTING-H1 provides protection comparable to subcutaneous administration of BCG. Applicant's results indicate that NanoSTING adjuvanted protein vaccines can elicit a multi-factorial immune response that protects from infection by Mycobacterium tuberculosis (M. tuberculosis).

Without being bound by theory, Applicant anticipates that the liposomal formulation offers two advantages. First, since 2′3′-cGAMP is rapidly degraded by ENPP-1 and has a half-life of ~30 min in vivo, encapsulation inside nanoparticles can lead to improved half-life and bioavailability. Second, as Applicant have previously demonstrated, liposomal nanoparticles facilitate the decoration of multiple copies of the protein antigen on the liposomal surface, leading to enhanced immunogenicity. As such, this Example suggests that NanoSTING is a potent mucosal adjuvant against Mtb that warrants further investigation with next-generation TB antigens, which might yield even better protection than the current formulation.

### Example 1.1. Plasmid Design

The protein sequence encoding Mtb-secreted antigen Ag85b (Accession Code: AY207396.1) and ESAT-6 (Accession code: AF226277.1) were downloaded from Genbank. The H1 antigen gene block was created at IDT (Coralville, Iowa), and Applicant optimized the codons for expression in Escherichia coli. Applicant used Gibson Assembly to insert the gene block sequence, which included the Sumo tag and the full-length H1 antigen gene, under the control of a T7 promoter in the plasmid backbone of the pET28A(+) plasmid after cutting it at the NdeI and XhoI restriction sites. Electroporation on electrocompetent E. coli EC10 cells was used for bacterial transformation and plasmid propagation.

### Example 1.1.1. Protein Expression and Purification

Briefly, E. coli BL21(DE3) cells were transformed with the plasmid encoding recombinant Sumo-H1 Ag protein. Cells were seeded in 500 mL of Luria-Bertani broth and cultured at 37° C. until the OD₆₀₀ reached 0.6. The next step was stimulating Sumo-H1 Ag expression using 1 mM IPTG for 5 hours. Cells were harvested and suspended in 20 mL buffer A (100 mM NaH₂PO₄, 10 mM Tris-Cl, 8 M Urea [pH 8]) and lysed by sonication: four cycles (each at 10 s on at 30% amplitude, 20 s off, at 4° C.). Urea in buffer A solubilized the inclusion bodies. The lysate was then centrifuged at 20,000×g for 30 min at 4° C. to remove cell debris and subjected to immobilized metal affinity chromatography (IMAC).

For IMAC purification, the lysate was loaded onto a gravity flow column with Ni-Sepharose High-Performance resin, which had been previously equilibrated for 20 min with 20 mL Buffer A. The column was then washed with 10 CV of Buffer B (100 mM NaH₂PO₄, 10 mM Tris-Cl, 8 M Urea [pH 6.3]), and the bound proteins were eluted with Buffer C (100 mM NaH₂PO₄, 10 mM Tris-Cl, 8 M Urea [pH=5.9]).

The eluted protein was dialyzed against Factor Xa digestion buffer (20 mM Tris, 50 mM NaCl, 100 mM Urea, 1 mM CaCl₂, pH=8.0) and then incubated with Factor Xa overnight at 4° C. to remove the Sumo tag. The cleaved protein was then dialyzed against sterile PBS to precipitate the H1 antigen selectively while the Sumo tag remained soluble. The subsequent steps were performed using sterile reagents and endotoxin-free labware to keep endotoxin to a minimum. The precipitated protein was then centrifuged at 10,000×g for 10 min to form a pellet, and the pellet was redissolved in 8 M urea solution to solubilize the protein. Finally, the soluble protein was dialyzed against 50 mM L-Arg, 50 mM L-Glu, 50 mM Na₂HPO₄, 0.05% Tween-20, pH=8.5, and stored at −20° C. until further use. Applicant tested the endotoxin level in the final protein solution using Pierce™ LAL Chromogenic Endotoxin Quantitation Kit (ThermoFisher Scientific, MA, USA) and confirmed that the total amount of endotoxin delivered intranasally was <5 EU per mouse.

### Example 1.1.2. Preparation of NanoSTING and Vaccine Formulation

Preparation and characterization of cGAMP encapsulation liposomes were described previously. Briefly, the liposomes were synthesized with a molar ratio of 10:1:1:1 for DPPC, DPPG, Cholesterol (Chol), and DPPE-PEG2000. Applicant combined the lipids with CHCl₃ and CH₃OH to create the liposomes. Then, Applicant evaporated the mixture using a vacuum rotary evaporator for around 80 min at 45° C. Applicant dried the resulting lipid thin film until all organic solvents evaporated. Applicant added a pre-warmed cGAMP solution (0.3 mg/mL in PBS buffer at pH 7.4) to hydrate the lipid film. The hydrated lipid films were mixed at 65° C. and subjected to freeze-thaw cycles. Applicant sonicated the mixture with a Branson Sonicator for 60 min (40 kHz). The free untrapped cGAMP was removed by Amicon Ultrafiltration units (MW cut off 10 kDa). Applicant used PBS buffer to wash the cGAMP-liposomes three times thoroughly. Using a calibration curve for cGAMP at 260 nm, the Take3 Micro-Volume absorbance analyzer of Cytation 5 (BioTek) was used to determine the amount of cGAMP in the filtrates. By deducting the quantity of free cGAMP in the filtrate, Applicant determined the final concentration of liposomal-encapsulated cGAMP and encapsulation efficiency.

Applicant mixed H1 antigen with the NanoSTING suspensions at room temperature for 10 min to allow the protein to adsorb onto the liposomes. The formulated vaccine was stored at 4° C. and used for up to two months. Applicant stored the NanoSTING suspensions at 4° C., and they were stable for 11 months. The average particle diameter, polydispersity index, and zeta potential were characterized at room temperature by the Litesizer 500 (Anton Paar).

### Example 1.1.3. Bacterial Strains and Culture

Mtb Erdman and M. bovis BCG were cultivated at 37° C. in 1 L roller bottles containing 100 mL of Middlebrook 7H9 (Difco) broth supplemented with 10% (v/v) Oleic-Albumin-Dextrose-Catalase (OADC), 0.2% (v/v) glycerol, and 0.05% (v/v) Tween-80. For quantification of colony-forming-units (CFUs), bacterial suspension and tissue homogenates were spread onto solid Middlebrook 7H10 agar medium supplemented with 10% (v/v) OADC, 0.5% (v/v) glycerol, and 100 μg/mL cycloheximide. Mycobacteria were grown at 37° C. for 3-4 weeks.

### Example 1.1.4. Mice Immunization and Mtb Challenge

For the immunogenicity study, groups of six- to ten-week-old female C57BL/6 mice were immunized intranasally with groups: 1) PBS, 2) 10 μg H1 antigen, and 3) 10 μg H1 antigen+20 μg NanoSTING. For all the groups, Applicant administered 20-25 μL of the appropriate formulation. Four weeks after the initial immunization, the animals were boosted with the corresponding first dosage. Six weeks from the day of the first dosage, the animals were euthanized, and lung and spleen samples were collected for ELISPOT and ICS assays.

Groups of five-week-old female C57BL/6 mice were obtained for one week before immunization. Six weeks before the challenge, two groups of mice (n=26) were anesthetized by isoflurane inhalation for three minutes. Then, either the vaccine formulation or PBS alone as an unvaccinated control was applied to the nares for inhalation. At two weeks pre-challenge, mice received a booster as described above. As a positive control, one group of mice (n=26) was injected subcutaneously once with approximately 5×10⁵ CFU of M. bovis BCG in a volume of 200 μl of PBS at six weeks pre-challenge. One week before the Mtb challenge, all the mice were moved to an ABSL-3 room for acclimation. Applicant aerosolized an Mtb suspension at OD₆₀₀ of 0.2 on day 0 to infect the mice. Applicant sacrificed six animals on day 1 and determined the average bacterial burden to be 500 CFU. The health of the animals was monitored daily, and body weight was measured weekly.

### Example 1.1.5. Organ Processing Post-Challenge CFU

At 3- and 10 weeks post-exposure, groups of mice (n=4-5) were sacrificed for CFU determination in lung, spleen, and liver homogenates and histological analyses. For CFU determination, organs were homogenized in gentleMACS C tubes (Miltenyi Biotec) with 2 mL of PBS. Serial dilutions of the homogenates were spread onto 7H10-OADC agar media containing 100 μg/mL cycloheximide, and plates were incubated at 37° C. for at least three weeks. For histology, the lungs were inflated through the trachea with 2-3 mL of 10% neutral-buffered formalin (NBF) before dissection. Organs were stored at 4° C. in 10% NBF. For antigen recall and flow cytometry analyses, a group of mice (n=5) were euthanized at 4- and 10 weeks post Mtb exposure. Lung and spleen tissues were collected, diced, and homogenized in gentleMACS C tubes, following Milteny Biotec's protocols for Mouse Lung and Spleen Dissociation, respectively. Erythrocytes were removed by washing the cell pellets with 1× Red Blood Cell Lysis buffer (Miltenyi Biotec). Cells were enumerated with Trypan Blue (0.04%) exclusion using a hemocytometer. Washed cells were resuspended at a density of 1-2.0×10⁷ cells/mL in RPMI 1640 (Corning) containing 10% fetal bovine serum (FBS) (Corning) (R10) and freshly supplemented with 50 μM 2-mercaptoethanol (Sigma Aldrich) and 1% (w/v) penicillin-streptomycin (Gibco).

### Example 1.1.6. Processing of Spleen and Lungs for ELISPOT

Applicant utilized 5 mL of 0.1 mM EDTA in PBS without Ca²⁺ or Mg²⁺ to perfuse the lung vasculature and injected it into the right cardiac ventricle to isolate lung cells. Each lung was divided into 100-300 mm² pieces with a knife. Applicant transferred the minced lung tissue in a digestion buffer containing collagenase D (2 mg/mL, Roche) and DNase (0.125 mg/mL, Sigma) in 5 mL of RPMI. The tissue was incubated in the digestion buffer at 37° C. for 1 hour and 30 minutes while vortexing every 10 minutes. Applicant disrupted the remaining intact tissue by passage (6-8 times) through a 21-gauge needle. The reaction was then put on hold by adding 500 μL of an ice-cold reaction-stopping buffer (lx PBS, 0.1 M EDTA). Next, Applicant passed the suspension through a 40 μm disposable cell strainer (Falcon) to remove large tissue pieces and collected the single cells in a 50 mL tube. The cells were centrifuged at 600×g for 10 min to form a pellet. The red blood cells (RBCs) were subsequently lysed by resuspending the cell pellet in 3 mL of ACK Lysing Buffer (Invitrogen), then centrifuged again at 600×g for 10 min. After the supernatants were discarded, the cell pellets were resuspended in 5 mL of full RPMI media (Corning). Next, Applicant stored the spleens in RPMI medium until they were ready for processing. Spleens were homogenized by pushing them through a 40 μm cell strainer using the hard end of a syringe plunger. Then, Applicant removed RBCs from splenocytes by incubating them in 3 mL of ACK lysis solution for 3 min at room temperature. Finally, Applicant passed the mixture through a 40 μm strainer to get a single-cell suspension. Applicant used the trypan blue exclusion method to count the number of splenocytes and lung cells.

### Example 1.1.7. ELISPOT

Applicant used the Mouse IFNγ ELISPOT Basic Kit (ALP) to perform the IFNγ ELISPOT according to the manufacturer's instructions (Mabtech, VA, USA). Applicant incubated the ELISPOT plates (Ref: MSIPS4W10, Millipore) with AN18 IFNγ (1 μg/mL, Mabtech #3321-3-250) overnight coating antibody at 4° C. The plates were washed five times with sterile PBS before adding lung lymphocytes or splenocytes. Cells from each animal were treated in four different groups: 1) negative control (R10 medium), 2) positive control (10 ng/mL phorbol 12-myristate 13-acetate (PMA) (Sigma, St. Louis, MT, USA) and 1 p g/mL of ionomycin), 3) Ag85b peptide pool (1.4 g/mL/peptide, JPT, Germany) and 4) ESAT-6 peptide pool (2 g/mL/peptide, BEI Resources, VA, USA). Lung lymphocytes and splenocytes were added to the plate in triplicates, where 3×10⁵ cells were added for negative controls, Ag85b, and ESAT-6 peptide pool stimulation. For positive control stimulation, Applicant only added 1×10⁴ cells. The cells were stimulated for 16 hours. Applicant washed off the cells the following day and incubated the plates with a biotinylated R4-6A2 anti-IFNγ- (Mabtech #3321-6-250) detection antibody. Following the wash, Applicant treated the wells for 1 hour at room temperature with diluted Extravidin-ALP conjugate (1:30,000). (Sigma, St. Louis, MI, USA). Then, Applicant added 70 μL/well of BCIP/NBT-plus substrate (Mabtech #3650-10) to the plate to produce the spots. Finally, the substrate was rinsed off with water after 20-30 min incubation. The spots were measured using Cytation 7 (BioTek Instruments, Inc.). Every spot represents a different cytokine-secreting cell. Applicant displayed the values as the average of measured triplicates with the background eliminated.

### Example 1.1.8. Post-Challenge Intracellular Cytokine Staining

Antigen recall, staining, and flow cytometry analysis determined ESAT-6 and Ag85b-specific lung and spleen T-cell responses. In 96-well round-bottom plates, cells (1-2×10⁶ lung or spleen cells/well) were aliquoted and stimulated at 37° C., 5% CO₂ for 12 hours with 1.4 g/mL of Ag85b and 2 μg/mL ESAT-6 peptides. As positive and negative controls, cells were incubated with PMA (10 ng/mL), ionomycin (1 μg/mL), or media alone. To allow cytokine accumulation, 50 μL of GolgiStop (BD Biosciences) diluted 1:500 in R10 medium was added to each well and further incubated at 37° C., 5% CO² for 4 hours. Cells were pelleted by centrifugation at 300×g for 5 min, and supernatants were collected and kept frozen at −80° C. for cytokine profiling.

For labeling, cells were washed with PBS and then incubated with Live/Dead Zombie Green (Biolegend; cat. no 423112). Fc receptors were blocked with unlabeled anti-mouse CD16/CD32 (clone 2.4G2; BD Biosciences). After washing with FACS buffer (PBS+2% FBS), cell surface markers were stained with anti-mouse CD3-PerCPCy5.5 (Biolegend; cat. no 100218), CD4-PE-Cy7 (Biolegend; #100422), CD8-Spark Blue 574 (Biolegend; cat. no 100794), KLRG1-BV510 (Biolegend; cat. no 138421), and CXCR3− Alexa Fluor 700 (Biolegend; cat. no 353742). Applicant fixed the cells with BD CytoFix/Perm and washed them thoroughly with BD Perm/Wash solution.

Intracellular cytokines were labeled with anti-mouse IFNγ-APC (Biolegend; cat. no 505810) and IL17-Pacific Blue (Biolegend; cat. no 506918) prepared in BD Perm/Wash solution, and then the cells were washed with Perm/Wash buffer. Cells were stained with the Live-Dead Zombie Green for compensation controls and labeled with the same panel of antibodies, individually or in FMO combinations. Samples were finally transferred into 5-mL polystyrene round-bottom test tubes with a cell strainer snap cap (Corning). Data was acquired on a BD LSRFortessa X-20 flow cytometer apparatus and analyzed using FlowJo™ v10.8 Software (BD Life Sciences).

### Example 1.1.9. Biodistribution Studies

Applicant used fluorescently labeled H1 antigen as a distribution marker to observe the effect of liposomal adjuvants on the residence time of the protein antigen. The iFluor 647 maleimide dye (AAT Bioquest, Pleasanton, CA) was conjugated to H1 antigen according to the manufacturer-recommended protocol. Next, Applicant intranasally administered 25 μL of a mixture of H1 antigen-iFluor 647 and NanoSTING (12.5 μL in each nostril) into BALB/c mice. The biodistribution of the dye was visualized at different time points after immunization using an in vivo imaging system (PerkinElmer, Waltham, MA). The fluorescence strength was quantified within regions of interest using the ImageJ (Easter Greenbush, NY) image processing program.

### Example 1.1.10. ELISA

Applicant tested the magnitude of vaccine-induced antibody response in serum against Ag85b using ELISA. In short, Applicant incubated high protein binding ELISA plates (Corning, NY, USA) with Ag85b protein (Abcam, MA, USA) at 0.25 μg/mL in phosphate-buffered saline (PBS) overnight at 4° C. or for 2 hours at 37° C. Applicant washed the plates with PBS+0.05% Tween20 (PBST) to remove unbound protein. The plates were blocked with PBS+1% BSA (Fisher Scientific, PA, USA)+0.1% Tween20 for 2 hours at room temperature. Following three more washes with PBST, Applicant added the serum samples at different dilutions to the plate. Applicant washed the plates with PBST and added HRP-conjugated anti-mouse IgG (Jackson ImmunoResearch Laboratories, 1:20000; PA, USA). Finally, Applicant developed the plates using 1-Step™ TMB ELISA substrate (ThermoFisher Scientific).

### Example 1.1.11. Histopathology

Lungs fixed in 10% neutral buffered formalin were processed and embedded in paraffin blocks, cut as 5 m sections, and slides stained with hematoxylin and eosin using routine histology methods. Slides were scanned on a Vectra Polaris scanning microscope (Akoya Biosciences, Marlborough, MA) using the brightfield setting at 20× magnification. These digital images were analyzed using Visiopharm image analysis software (Visiopharm, Westminster, CO), employing a custom designed algorithm for the detection of tuberculosis pathology in lung tissue. Briefly, for each tissue section, a region of interest (ROI) identification algorithm was generated at a low magnification with custom decision forest training and classification to differentiate tissue versus background based on color and area. Tissue lesions were identified within tissue ROIs using a formulated K-means clustering algorithm based on staining intensity, area, and morphological features of inflammation. Lesion burden was expressed as a percent ratio between area of TB lesion pathology and total lung tissue area.

### Example 1.1.12. Statistical Analysis

All data are presented as mean values, and error bars represent SEM. Applicant performed all statistical analyses using GraphPad Prism (v8). Applicant used the Mann-Whitney t-test for pairwise comparisons. To compare multiple groups, Applicant used Dunn's multiple comparison test. Applicant performed Tukey's multiple test for repeated measures analysis to compare bodyweight and bacterial load data at each time point.

### Example 1.2. H1 Antigen Protein Expression and Vaccine Formulation

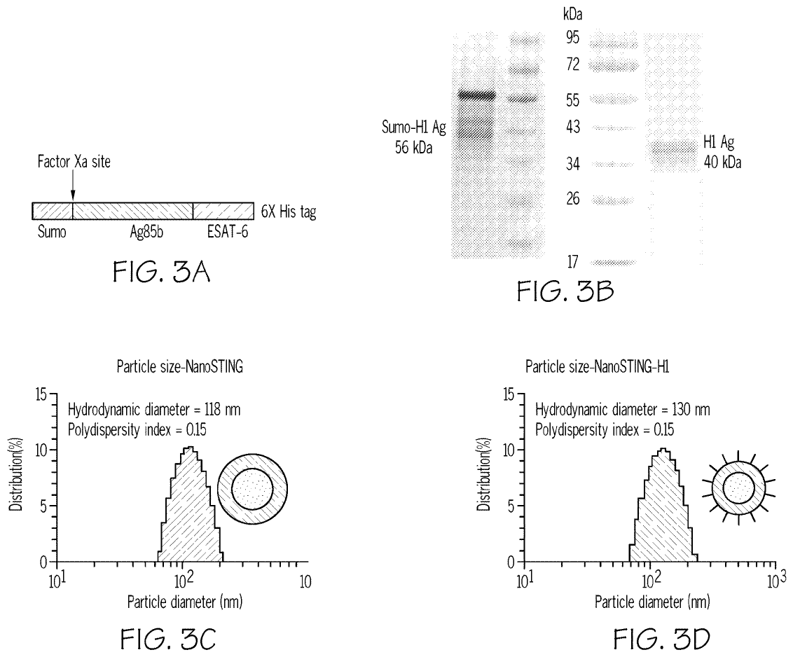

H1 is a synthetic fusion protein that include two different Mtb antigens and has been tested in humans. To enhance the solubility and expression of the H1 antigen, Applicant fused a Sumo-tag to the N terminus of the protein with a Factor Xa cleavage site between them (FIG. 3A). The protein was purified using standard Ni²⁺-NTA chromatography and the Sumo-tag was removed by factor Xa proteolysis. However, upon proteolysis, the H1 antigen rapidly precipitated out of solution. To ensure that Applicant has soluble protein for immunization, Applicant resolubilized the protein using 8 M urea, followed by dialysis of the resulting protein in a buffer containing known protein stabilizers (50 mM L-Arg and 50 mM L-Glu). Applicant's expression system using the Sumo-tag yielded ~40 mg/L of purified protein (FIG. 3B).

Applicant used a single-step “mix and immunize” approach to prepare the final vaccine formulation NanoSTING-H1, as described previously. X. An el al., “Single-dose intranasal vaccination elicits systemic and mucosal immunity against SARS-CoV-2,” iScience, vol. 24, no. 9, p. 103037, September 2021, doi: 10.1016/j.isci.2021.103037. As H1 antigen aggregates rapidly in PBS, Applicant sough to test if the NanoSTING-H1 formulation was stable for intranasal vaccination. Applicant utilized dynamic light scattering (DLS) to compare particle sizes of the nanoparticle adjuvant and protein mixture. Both NanoSTING and NanoSTING-H1 showed similar particle sizes, 118±1.6 nm (mean±SEM) and 130±1.8 nm, respectively, indicating no rapid protein aggregation in the vaccine formulation, at least during short-term incubation (30 min-1 hour) at 25° C. (FIGS. 3C-3D).

### Example 1.2.1. Intranasal Immunization with NanoSTING Increases the Retention Time of the Immunogen in the Nasal Cavity

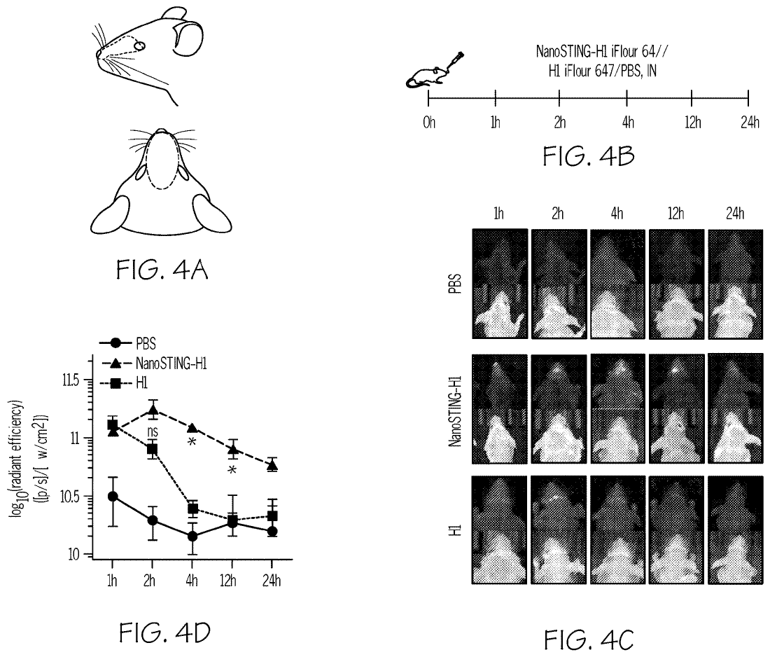

In addition to stabilizing the protein and increasing the avidity of display, liposomal formulations can increase the antigen's residence time within the mucosal compartment, facilitating a robust immune response. Typical protein antigens administered intranasally have a short residence time in the nasal cavity and are cleared in less than 1 hour. Therefore, Applicant aimed to test whether the developed liposomal adjuvant formulation can delay antigen clearance from the immunization site. To track the antigen in vivo, Applicant conjugated the purified H1 antigen with iFluor 647. Applicant then administered H1 iFluor 647 to groups of BALB/c mice, both with or without NanoSTING as an adjuvant and imaged the animals for 24 hours using an IVIS Spectrum system. Applicant tracked the antigen by quantifying the total fluorescence signal in a defined region of interest, capturing the mouse nasal cavity (FIG. 4A). Longitudinal profiling of the fluorescent H1 antigen in the nasal area of live animals revealed that, while the unadjuvanted H1 iFluor 647 was cleared in <4 hours, the NanoSTING adjuvanted H1 iFluor 647 was detectable up to 24 hours (FIGS. 4B-4D). These data suggest that the NanoSTING adjuvanted vaccine has a high residence time in the nasal cavity, thus promoting the likelihood of efficient antigen uptake and processing.

### Example 1.2.2. Intranasal Vaccination of Mice with NanoSTING-H1 Induces Cellular Immunity in the Lung and Spleen

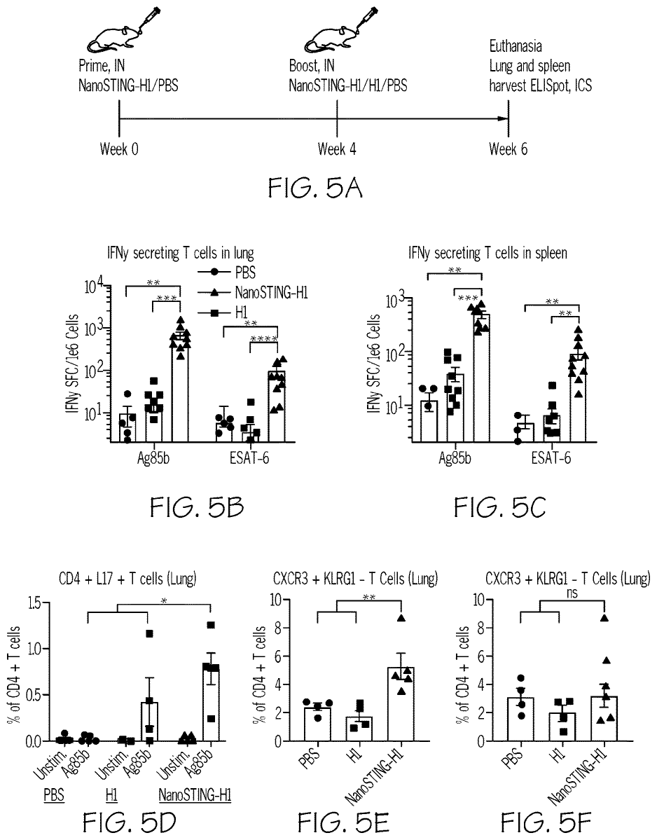

To evaluate the immunogenic potential of Applicant's experimental vaccine, groups of C57BL/6 mice were vaccinated following a prime/boost vaccination regimen, receiving two doses of (1) NanoSTING-H1, (2) H1 only, and (3) PBS four weeks apart (FIG. 5A). The animals were euthanized two weeks after the booster dose, and lung and spleen samples were harvested and processed to isolate single cells. Enzyme-linked immunospot (ELISPOT) assay was performed using the lung and spleen cells to determine if NanoSTING-H1 elicits Th1 immunity, as Th1 cytokine-secreting T cells are essential for the control of Mtb infection. NanoSTING-H1 induced antigen-specific IFNγ-secreting T cells in the lungs of vaccinated animals (Ag85b: 670±127 SFC/10⁶ cells, ESAT-6: 98±30 SFC/10⁶ cells), which was significantly higher when compared to H1 (Ag85b: 16±5 SFC/10⁶ cells, p-value<0.001; ESAT-6: 3±2, SFC/10⁶ cells, p-value<0.0001) and unvaccinated groups (Ag85b: 9±5 SFC/10⁶ cells, p-value=0.005; ESAT-6: 6±1 SFC/10⁶ cells, p-value=0.004) (FIG. 5B). Similarly in spleen, vaccination with NanoSTING-H1 resulted in significantly higher antigen-specific T cells (Ag85b: 520±63 SFC/10⁶ cells, ESAT-6: 90±23 SFC/10⁶ cells) compared to H1 (Ag85b: 39±11 SFC/10⁶ cells, p-value<0.001; ESAT-6: 5±3 SFC/10⁶ cells, p-value<0.001) and unvaccinated (Ag85b: 13±5 SFC/10⁶ cells, p-value=0.0014; ESAT-6: 5±2 SFC/10⁶ cells, p-value=0.0097) groups (FIG. 5C).

Besides Th1 responses, it has been previously demonstrated that a higher frequency of IL-17 secreting CD4⁺ T cells (Th17) in the lung can confer robust protection against virulent Mtb. To quantify the Th17 responses in the lung, Applicant performed intracellular cytokine staining (ICS) and flow cytometry. These data reveal that intranasal vaccination with NanoSTING-H1 induced significantly higher IL-17 secreting CD4⁺ T cells (0.78%±0.16%) in the lung when compared to the other two groups of animals (0.43%±0.26% for H1, and 0.07±0.04% for unvaccinated animals, p-value=0.03) (FIG. 5D).

A CXCR3⁺KLRG1⁻ lung parenchyma homing T cell population that shows an improved ability to control lung Mtb infection has been identified. Using flow cytometry, Applicant confirmed that vaccination with NanoSTING-H1 resulted in 5.3%±0.9% CXCR3⁺KLRG1⁻ parenchymal CD4⁺ T cells in the lung, which was significantly higher than protein-only (H1) group (1.84%±0.42%, p-value=0.015) (FIGS. 5E and 5F). Additionally, antigen-specific antibodies may enhance protection against pulmonary TB.

Additionally, vaccination with NanoSTING-H1 induced Ag85b specific IgG in serum (mean endpoint titers, NanoSTING-H1: 750±350, unvaccinated: 50) that increased after the booster dose (mean endpoint titers, NanoSTING-H1: 3800±3000, unvaccinated: 50). In summary, NanoSTING-H1 induces Th1-specific cellular immunity, IL-17⁺ and CXCR3⁺KLRG1⁻ CD4⁺ T cells in the lung, and, antigen-specific humoral immunity that may have a synergistic effect in protection against pulmonary TB.

### Example 1.2.3. NanoSTING-H1 Vaccination Protects Mice Against Challenge with Virulent Mtb Strain

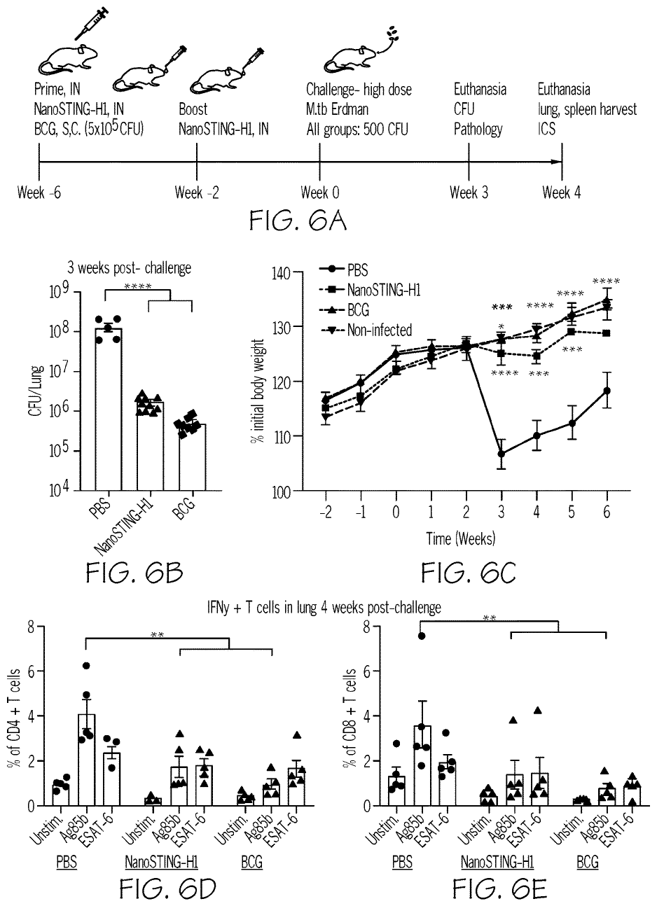

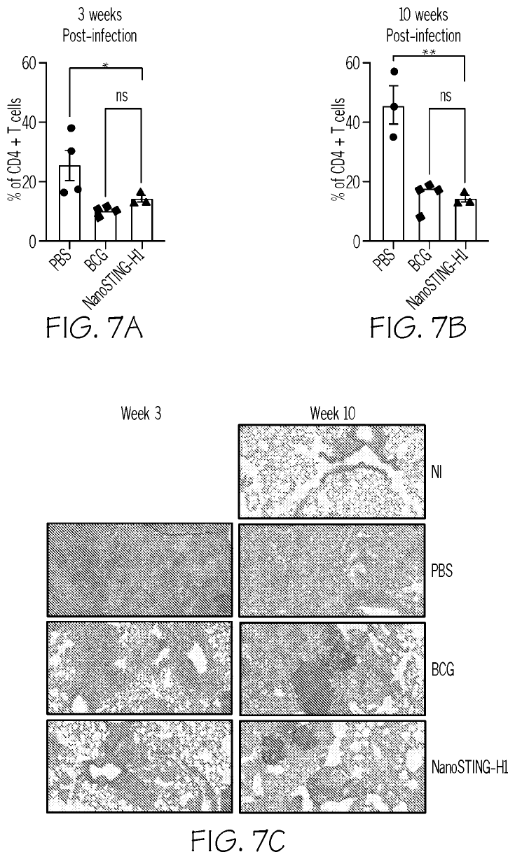

Next, Applicant aimed to determine if the developed vaccine was protective against live Mtb infection. Groups of C57BL/6 mice were vaccinated intranasally with the same prime-boost regimen (FIG. 6A). The vaccinated animals were subsequently challenged with aerosolized M. tb Erdman. Applicant also vaccinated 10 animals with a single dose of subcutaneous BCG vaccine six weeks before the challenge as a comparative positive control (FIG. 6A). As expected, unvaccinated mice showed a significantly higher bacterial burden in the lung (1.30±0.3×10⁸ CFU, p-value<0.0001) compared to either the NanoSTING-H1 (1.6±0.2×10⁶ CFU) or BCG (5.3±0.6×10⁵ CFU) vaccinated animals (FIG. 6B). NanoSTING-H1 and BCG vaccination also limits bacterial replication in the liver and spleen over at least four weeks. The reduced bacterial burden in mice corresponds to significantly reduced mean peak weight loss in the NanoSTING-H1 group (1.4±0.5%), compared to unvaccinated animals (mean peak weight loss of 15.3±1.7%) (FIG. 6C). Finally, Applicant studied the Th1 T cell response in lung following Mtb challenge. Consistent with the expectation that vaccination-induced immunity reduces the bacterial burden and could lower the T-cell responses during immediate challenge, both vaccinated groups BCG (Ag85b: 1.0±0.2%, ESAT-6: 1.7±0.4%) and NanoSTING-H1 (Ag85b: 1.8±0.5%, ESAT-6: 1.8±0.3%) did not enhance the frequency of CD4⁺IFNγ⁺ T cells beyond the infection-induced (Ag85b: 4.0±0.6%, ESAT-6: 2.4±0.2%, p-value=0.0045) values (FIG. 6D). A similar observation was also true for the CD8⁺ T cells; ICS on the lung cells revealed similar frequencies of antigen-specific CD8⁺ T cells in the lungs of BCG (Ag85b: 0.8±0.2%, ESAT-6: 0.9±0.2%) and NanoSTING-H1 (Ag85b: 1.4±0.6%, ESAT-6: 1.5±0.7%) vaccinated animals. Both were significantly lower than infection-induced (Ag85b: 3.7±1%, ESAT-6: 2±0.3%) levels (FIG. 6E).

Lastly, Applicant evaluated the lung immunopathology three- and 10-weeks post-challenge to detect areas of inflammatory lesions on hematoxylin and eosin (H&E) stained tissue sections from the accessory lung lobes. Both BCG and NanoSTING-H1 groups had significantly lower mean lung lesion areas at three (mean lesion percentage: BCG: 9.7±0.6% and NanoSTING-H1: 13.4±0.7%) and 10 weeks (BCG: 17.7±0.4% and NanoSTING-H1: 14.1±0.9%) after challenge, quantified as the percentage of lesions, compared to the unvaccinated control group (3 weeks post-challenge: 25±4.5%, p-value=0.0493; 10 weeks post-challenge: 46±5.3%, p-value=0.0021). Therefore, intranasal administration of NanoSTING-H1 induces robust immunity in vaccinated animals and protects Mth challenged animals from severe weight loss and inflammatory tissue damage by reducing bacterial burden in lung and other vital organs.

### Example 1.3. Discussion

New and efficient immunization strategies are required due to the BCG's limitations in protecting adults and immunocompromised individuals against pulmonary TB. Owing to considerable safety concerns posed by live-attenuated vaccines, early alternative attempts at an improved TB vaccine included killed bacilli, and mycobacteria cell extract vaccines. However, instead of providing long-term protection, these vaccines showed an inclination towards inducing a ‘hypersensitive’ non-specific inflammatory response. Another approach towards a safe and potent TB vaccine is considered secretory or total cell culture protein (CFP)-based vaccines. Despite showing noticeable protective efficacy in animal models, these vaccines present a complex repertoire of protein antigens, which subsequently leads to suboptimal immune response due to competition for antigen presentation. Subunit vaccines can be tailored to recognize key Mtb antigens, offering greater control over the specificity of the immune response. As the immunogens used in subunit vaccines are purified and non-essential elements are excluded, these vaccines pose a much lower risk of unwanted inflammatory responses. Several subunit vaccines have been tested in animal models with attractive safety profiles, and currently, a few of them are in clinical trials: M72/AS01E, ID93+GLA-SE, H56/IC31, and GamTBvac. The principal route of administration for all TB vaccines currently in trials is via injection intramuscularly or intradermally. However, as the lung is where Mtb infection first manifests, administering a TB vaccine intranasally can boost both mucosal and peribronchial immune responses against Mtb.

In this Example, Applicant explored the efficacy of cGAMP, a STING-agonist, as an adjuvant in an intranasal vaccine that induces Th1-specific response, lung parenchyma homing T cells in vaccinated animals and provides protection against virulent Mtb. The H1 antigen fusion protein and NanoSTING together protected from challenge with virulent Mtb. Experimental vaccinations with NanoSTING adjuvant provided protection that inhibited bacterial replication in the spleen and liver for at least four weeks following the challenge.

The H1 antigen protein Applicant utilized contains two well-characterized immunodominant proteins that induce significant T cell responses: Ag85b and ESAT-6. IFNγ secreted by CD4⁺ T cells is essential for infection control, host survival, and long-term reduction of bacterial burden. IFNγ knockout mice succumb to the Mtb challenge, as they fail to control bacterial replication compared to control animals. Data from macaques also show that CD4⁺ T cells may contain rapid TB progression and early bacterial dissemination from the site of infection to other organs. Additionally, IFNγ secreted by CD4⁺ T cells facilitates a robust CD8⁺ T cell response.

NanoSTING, as a mucosal adjuvant, enhances IFNγ-secreting T cell response conferred by the antigens in the lung and spleen. Applicant observed Ag85b specific CD4+ IFNγ-secreting Th1 T cells in the spleen of animals receiving NanoSTING-H1, which may explain decreased bacterial load in the spleen and liver four weeks post-challenge. Besides Th1 cells, the development of Th17 cells was likely related to the enhanced performance of intranasal NanoSTING-H1. While Th17 cells were shown to be dispensable for primary immunity to Mtb, they were correlated with enhanced protection against highly virulent Mtb strains. IL-17 was also correlated with the rapid recruitment of IFNγ-producing T cells in the lung upon infection and was required for the full efficacy of a BCG vaccine.

Applicant observed CD4⁺ CXCR3⁺ KLRG1⁻ T cells in the lungs of vaccinated animals, which are known to migrate to lung parenchyma and control bacterial infection. The loss of BCG-induced protection was correlated with increased KLRG1-expressing T cells. This is encouraging data, as multiple studies have suggested that KLRG1⁻CD4⁺ T cells have enhanced proliferative ability and routinely replicate to replenish T cell protection at the site of infection.

The role of antibody-mediated immunity against TB remains controversial. Early studies with B cell-knockout mice indicated that antibody response against Mtb antigens was disposable in protection against pulmonary TB. However, a recent clinical trial with a protein-based vaccine suggested that TB-specific antibodies may play a role in protecting high-risk BCG-primed individuals against sustained Mtb infection

For intracellular pathogens, cyclic dinucleotides (CDN) offer much potential as a vaccine adjuvant. Natural CDNs can prime innate immunity through the cGAS-STING pathway. It has recently been demonstrated that a STING-agonist cyclic dinucleotide adjuvant is effective in a protein-based intranasal vaccine against TB. Their adjuvant ML-RR-cGAMP was modified to include phosphorothioate internucleotide linkages, which protects it from hydrolysis by phosphodiesterases.

However, Applicant took advantage of nanoparticles to encapsulate and deliver cGAMP to the antigen-presenting cells, bypassing the problem of extracellular hydrolysis. The endogenous 2′-3′ cGAMP, the adjuvant in Applicant's vaccine, exhibits higher binding affinity to STING among cGAMP molecules containing other phosphodiester linkages. Additionally, liposomal NanoSTING increases the residence time of H1 antigen in the nasal cavity, which may explain the need for only a single booster dose to yield BCG-comparable protection in the lung.

In sum, this Example shows that nanoparticles encapsulating 2′-3′-cGAMP are an effective adjuvant against virulent Mtb infection in mice. NanoSTING is a promising candidate for developing a safe and potent subunit vaccine for immunocompromised individuals. This preliminary work warrants further investigations into vaccine formulations with NanoSTING and next-generation TB antigens for enhanced protection against TB.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A composition comprising:
a particle, an antigen, and a modulator encapsulated within the particle,
wherein the modulator is selected from the group consisting of an agonist, an activator of the immune system, or combinations thereof, and
wherein the antigen is operable to elicit an immune response in a subject against Mycobacterium tuberculosis.

2. The composition of claim 1, wherein the modulator comprises an endogenous agonist of the stimulator of interferon genes (STING) pathway.

3. The composition of claim 1, wherein the modulator is selected from the group consisting of an endogenous agonist of the stimulator of interferon genes (STING) pathway, bis-(3′,5′)-cyclic dimeric guanosine monophosphate (c-di-GMP), cyclic guanosine monophosphate-adenosine monophosphate (cGAMP), 2′,3′-cyclic guanosine monophosphate-adenosine monophosphate (2′,3′-cGAMP), amidobenzimidazole, derivatives of amidobenzimidazole, nucleotide modulators, plasmid DNA modulators, CF501, MSA-2, SHR1032, C-178, H-151, divalent cations or combinations thereof.

4. The composition of claim 1, wherein the modulator comprises 2′,3′-cyclic guanosine monophosphate-adenosine monophosphate (2′,3′-cGAMP).

5. The composition of claim 1, wherein the antigen comprises a protein or peptide derived from Mycobacterium tuberculosis, or a nucleotide sequence encoding the protein or peptide.

6. The composition of claim 5, wherein the antigen is selected from the group consisting of Ag85b, ESAT6, Rv1813, Rv1733c, RV2626c, Rv2660c, RpfD, Ag85A, CFP10, TB10.4, EsxV, EsxW, MTB32A, MTB39A, PPE42, a full protein thereof, a protein fragment thereof, a peptide fragment thereof, a fusion protein thereof, or combinations thereof.

7. The composition of claim 1, wherein the antigen comprises a protein or peptide derived from Mycobacterium tuberculosis.

8. The composition of claim 1, wherein the antigen comprises a nucleotide sequence encoding a protein or peptide derived from Mycobacterium tuberculosis.

9. The composition of claim 1, wherein the antigen comprises a fusion protein of Mycobacterium tuberculosis, or a nucleotide encoding the fusion protein.

10. The composition of claim 9, wherein the fusion protein is selected from the group consisting of a fusion of two or more of Ag85b, ESAT6, Rv1813, Rv1733c, RV2626c, Rv2660c, RpfD, Ag85A, CFP10, TB10.4, EsxV, EsxW, MTB32A, MTB39A, PPE42, a full protein thereof, a protein fragment thereof, a peptide fragment thereof, or combinations thereof.

11. The composition of claim 1, wherein the antigen comprises a fusion protein of Ag85b and ESAT6.

12. The composition of claim 11, wherein the fusion protein comprises SEQ ID NO: 1 or a sequence with at least 65% sequence identity to SEQ ID NO: 1.

13. The composition of claim 1, wherein the antigen comprises a nucleotide sequence encoding a fusion protein of Ag85b and ESAT6.

14. The composition of claim 13, wherein the nucleotide sequence comprises SEQ ID NO: 2 or a sequence with at least 65% sequence identity to SEQ ID NO: 2.

15. The composition of claim 1, wherein the antigen is associated with an outer surface of the particle.

16. The composition of claim 1, wherein the composition comprises a lipid-based particle.

17. The composition of claim 16, wherein the lipid-based particle is in the form of a liposome.

18. The composition of claim 1, wherein the composition is suitable for use in treating or preventing tuberculosis.

19. A method of treating or preventing tuberculosis in a subject, said method comprising:
administering a composition to a subject, wherein the composition comprises:
a particle, an antigen, and a modulator encapsulated within the particle,
wherein the modulator is selected from the group consisting of an agonist, an activator of the immune system, or combinations thereof, and
wherein the antigen is operable to elicit an immune response in a subject against Mycobacterium tuberculosis.

20. The method of claim 19, wherein the method is used to prevent tuberculosis in the subject.

21. The method of claim 19, wherein the method is used to treat tuberculosis in the subject.

22. The method of claim 19, wherein the subject is a human being.

23. The method of claim 19, wherein the composition is administered through intranasal administration.

24. The method of claim 19, wherein the modulator comprises an endogenous agonist of the stimulator of interferon genes (STING) pathway.

25. The method of claim 19, wherein the modulator is selected from the group consisting of an endogenous agonist of the stimulator of interferon genes (STING) pathway, bis-(3′,5′)-cyclic dimeric guanosine monophosphate (c-di-GMP), cyclic guanosine monophosphate-adenosine monophosphate (cGAMP), 2′,3′-cyclic guanosine monophosphate-adenosine monophosphate (2′,3′-cGAMP), amidobenzimidazole, derivatives of amidobenzimidazole, nucleotide modulators, plasmid DNA modulators, CF501, MSA-2, SHR1032, C-178, H-151, divalent cations or combinations thereof.

26. The method of claim 19, wherein the modulator comprises 2′,3′-cyclic guanosine monophosphate-adenosine monophosphate (2′,3′-cGAMP).

27. The method of claim 19, wherein the antigen comprises a protein or peptide derived from Mycobacterium tuberculosis, or a nucleotide sequence encoding the protein or peptide.

28. The method of claim 19, wherein the antigen is selected from the group consisting of Ag85b, ESAT6, Rv1813, Rv1733c, RV2626c, Rv2660c, RpfD, Ag85A, CFP10, TB10.4, EsxV, EsxW, MTB32A, MTB39A, PPE42, a full protein thereof, a protein fragment thereof, a peptide fragment thereof, a fusion protein thereof, or combinations thereof.

29. The method of claim 19, wherein the antigen comprises a fusion protein of Ag85b and ESAT6.

30. The method of claim 29, wherein the fusion protein comprises SEQ ID NO: 1 or a sequence with at least 65% sequence identity to SEQ ID NO: 1.

31. The method of claim 19, wherein the antigen comprises a nucleotide sequence encoding a fusion protein of Ag85b and ESAT6.

32. The method of claim 31, wherein the nucleotide sequence comprises SEQ ID NO: 2 or a sequence with at least 65% sequence identity to SEQ ID NO: 2.

33. The method of claim 19, wherein the antigen is associated with an outer surface of the particle.

34. The method of claim 19, wherein the composition comprises a lipid-based particle.

35. The method of claim 34, wherein the lipid-based particle is in the form of a liposome.
